(19) United States
(12) Patent Application Publication
(10) Pub. No.: US 2005/0113606 A1
(43) Pub. Date: May 26, 2005

(54) Continuous production of chlorodifluoroacetyl fluoride via chlorotrifluoroethylene oxidation

(76) Inventors: Robert L. Bell, Amherst, NY (US); Timothy R. Demmin, Grand Island, NY (US); David E. Bradley, Buffalo, NY (US); David Nalewajek, West Seneca, NY (US)

Correspondence Address: Colleen D. Szuch, Esq.;Honeywell International Inc., 101 Columbia Road, Morristown, NJ, 07962-2245, US

(21) Appl. No.: 10/718,475

(22) Filed: Nov. 20, 2003

(51) Int. Cl. C07C 51/58

(52) U.S. Cl. 562/851

(57) ABSTRACT

The invention pertains to a process for preparing chlorodifluoroacetyl fluoride (CDAF) by oxidation of chlorotrifluoroethylene (CTFE) in a solvent using a continuously stirred tank reactor. It provides a process for the production of chlorodifluoroacetyl fluoride by comprises reacting a solvent solution of chlorotrifluoroethylene with oxygen in a reactor to form a product which comprises chlorodifluoroacetyl fluoride. The reacting may be conducted in a continuous or batch mode.

### BACKGROUND OF THE INVENTION

The present invention relates a process for preparing chlorodifluoroacetyl fluoride (CDAF) by oxidation of chlorotrifluoroethylene (CTFE) in a solvent using a substantially continuously stirred tank reactor.

Chlorodifluoroacetyl fluoride is a useful starting material or intermediate in the chemical syntheses of pharmaceuticals and agrochemicals. The active acid fluoride readily reacts with alcohols, amines and other bases to form the corresponding halogenated; ester, amide, acid and salts. The-oxidation of chlorotrifluoroethylene to form CDAF is known in the literature. Previous methods have suffered from a batch process limitation, poor yields and long reaction times. U.S. Pat. No. 2,676,983 pertains to a process for preparing chlorodifluoroacetyl fluoride by a batch reaction of neat CTFE, i.e. without any solvent. This process requires 12 to 22 hour reaction time and analysis of the material shows only a 40% to 50% yield. U.S. Pat. No. 3,536,733 shows a process which uses a batch or plug flow tubular reactor to make fully halogenated olefin epoxides in a solvent. Many of the solvents used in this process are no longer allowed by law, e.g. HFC-113, HFC-11, CCl₄. U.S. Pat. No. 3,959,367 describes a process for non-catalytic liquid phase oxidation of halo-olefins. It pertains to the formation of acid chlorides and no mention chlorodifluoroacetyl fluoride is made. This patent requires the use of high pressure, 500 psig to 1000 psig, to obtain good yields. When lower pressures are used long reaction time or low yields were obtained. Two reactors are required to complete the reaction with staged temperatures and pressures.

German Patent 947,364 uses CTFE for the formation of chlorotrifluoroethylene oxide rather than chlorodifluoroacetyl fluoride. More recent methods of preparing acid halides include the vapor phase continuous method of U.S. Pat. No. 5,919,341. This process contacts a halo-olefin with oxygen in the presence of a Cl₂ initiator to begin free radical addition. It also uses high energy UV to propagate the reaction and produces chlorodifluoroacetyl chloride rather than chlorodifluoro-acetyl fluoride. WO 96/29298 describes a process for preparing of polyfluoro-acetyl fluorides by oxidation. The method described in WO 96/29298 is run at supercritical pressures and temperatures and uses polyhalogenated alkyl substrates rather than perhalogenated olefins. U.S. Pat. No. 5,545,298 describes a process for preparing polyfluorocarboxylic acid chlorides and perfluorocarboxylic acid chlorides.

The process of this invention economically produces chlorodifluoroacetyl fluoride by feeding a solution of chlorotrifluoroethylene in suitable solvent to a reactor pressurized with oxygen at the proper reaction temperature. This production process can be operated in either batch or continuous modes. With this process, derivatives of CDAF can be economically produced with increased throughput, conversion, and selectivity.

### DESCRIPTION OF THE INVENTION

The invention provides a process for the production of chlorodifluoroacetyl fluoride which comprises reacting a solvent solution of chlorotrifluoroethylene with oxygen in a reactor to form a product which comprises chlorodifluoroacetyl fluoride.

The invention also provides a continuous process for the production of chlorodifluoroacetyl fluoride which comprises reacting a solvent solution of chlorotrifluoroethylene with gaseous oxygen with simultaneous agitation in a reactor to form a product which comprises chlorodifluoroacetyl fluoride.

The invention further provides a continuous process for the production of chlorodifluoroacetyl fluoride which comprises reacting a solvent solution of chlorotrifluoroethylene with gaseous oxygen with simultaneous agitation in a reactor to form a product which comprises chlorodifluoroacetyl fluoride; wherein the reaction is conducted by continually feeding the solvent solution of chlorotrifluoroethylene into the reactor, wherein the reactor is pre-pressurized with oxygen; and then subsequently removing chlorodifluoroacetyl fluoride from the product; wherein the solvent is selected from the group consisting of halogenated butanes, halogenated hexanes, dimethyl cyclobutanes, octadecafluorodecahydronaphthalene, and combinations thereof; wherein the chlorotrifluoroethylene concentration in the solvent ranges from about 1% to about 30% by weight; wherein the solvent solution is fed into the reactor at a rate which ranges from about 0.1 to about 3 times the reactor volume per hour; wherein if the reactor has vapor space, the amount of chlorotrifluoroethylene in the vapor space is maintained at less than 3 wt. %; wherein the reaction is conducted at a temperature in the range of from about 20° C. to about 200° C.; wherein the oxygen partial pressure is maintained in the range of from about 10 psia about to 300 psia; wherein the ratio of oxygen to chlorotrifluoroethylene ranges from about 0.01 to about 0.55 by weight.

As a first step in the production of chlorodifluoroacetyl fluoride, CTFE should preferably be pre-dissolved before it is put into the reactor. The solvent can be any solvent which effectively dissolves the chlorotrifluoroethylene. Non-limiting examples of the solvents claimed in this process include halogenated butanes, halogenated hexanes, dimethyl cyclobutanes, octadecafluorodecahydronaphthalene, and combinations thereof. Preferred solvents include perhalogenated butanes (C₄FₓCl_{y}) wherein x=(1 to 10) and y=(10-x); perhalogenated hexanes (C₆FₓCl_{y}) wherein x=(1 to 14) and y=(14-x); dimethyl cyclobutane (C₆FₓCl_{y}) wherein x=(1 to 12 ) and y=(12-x) and octadecafluorodecahydronaphthalene (C₁₀F₁₈). The CTFE concentration in the solvent may range from about 1% to about 30% by weight, with from about 4% to about 8% being preferred.

The reaction may be conducted in a continuous mode or a batch mode, however, a continuous mode is preferred. In a continuous process CDAF is produced continuously by adding the solution of CTFE in solvent into a well stirred reactor where oxygen is introduced at a controlled rate and pressure.

The reaction may be conducted in any suitable reaction vessel equipped for stirring or agitation, but it should preferably be constructed from materials which are resistant to corrosion such as nickel and its alloys, including commercially available Hastelloy, Inconel, Incoloy, and Monel or vessels lined with fluoropolymers. A preferred reactor is a 650 cc Hastelloy, Parr Autoclave equipped with agitator, thermocouple, dip tube, heating mantle, controller and cooling coils.

The general formulas for the product and side reactions are reaction are:

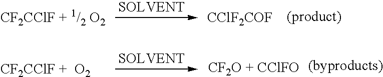

Solvent solution and reaction products are drained from the reactor to maintain a steady state. The volatile products/by-products are then flash distilled from the solvent, and the depleted solvent is then cooled, mixed with additional CTFE and then optionally recycled back into the reactor. The volatile flash distillate can then be used as is, or else fractionally distilled in a continual manner to yield recovered CDAF.

The continuously stirred tank reactor approach is the preferred method of synthesis. The specified temperatures, oxygen pressures, and the residence time should be controlled to yield product at usable quality and production rates. The solubility of the CTFE in the solvent is important to product selectivity and yield. The 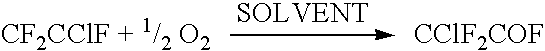
reaction occurs predominately in the liquid phase so product formation increases with CTFE solubility. As CTFE solubility in the solvent falls off, the formation of the byproduct by mainly the vapor phase reaction 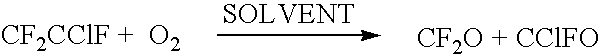
occurs. Minimization of the reactor head space also increases yield by reducing CTFE reactant in the vapor space. The maximum feed rate of the CTFE/solvent mixture is a function of the concentration of CTFE in the solvent. A balance is maintained between productivity and safe operating conditions. At an 8% solution of CTFE in solvent, the feed rates range from about 0.1 to about 3 times the reactor volume of exchanges per hour. For safety considerations and selectivity it desirable to keep the CTFE in the vapor space at <3 wt. % during steady state operations. The reaction rate increases with temperature. The preferred reaction temperature range is from about 20° C. to about 200° C. with the preferred range being from about 60° C. to about 110° C. The CTFE in the vapor is also a function of the temperature. As the temperature increases the solubility of the CTFE in the solvent is reduced with the resultant loss of selectivity because of increased byproduct formation in the vapor phase. Also at higher temperatures the increase partial pressure of CTFE in the vapor space approaches the lower flame limits. The concentration of O₂ in a solution is a function of the gas pressure over the surface of that liquid. A correlation between the partial pressure of O₂ over the CTFE/solvent mixture and the reaction time to completion was developed. The relationship is given by the expression
(240/O₂ Partial Pressure=Reaction Time)
where pressure is in psia and reaction time is in hours. Using 8% CTFE in C₆Cl₅F₉ solvent the expected reaction time is 16 hours at 15 psia; 8 hours at 30 psia; 4 hours at 60 psia; and 2 hours at 120 psia of O₂ partial pressure. For this reaction, the preferred oxygen partial pressure is in the range of from about 10 psia to about 300 psia with a preferred operating range of from about 60 psia to about 120 psia. With the correct O₂/CTFE ratio high productivity and selectivity are achieved while maintaining the vapor phase below the lower flame limits. The preferred ratio of oxygen to chlorotrifluoroethylene ranges from about 0.01 to about 0.55 by weight. Preferably the reactor is pre-pressurized with oxygen prior to introduction of the CTFE solution with simultaneous agitation.

This continuous process uses a two-phase vapor and liquid system. The high solubility of the CTFE in the solvent maintains the reaction in the liquid and provides good heat transfer to dissipate the (−56 Kcal/g-mole) of reaction heat. The limited mass transfer of the O₂ into the solvent prevents a runaway reaction. The relatively low concentration of the CTFE in the solvent retards the polymerization reaction. As neither reactants are in high concentrations in the solvent, peroxide formation is greatly suppressed, (<25 ppm). Since the system is mass transfer limited an increase in agitation rate results in an increased rate, but the reaction is stable over a large range of agitation rates. Due to the high reactivity of the system, high feed rates are possible which allow high throughput, good conversion, high selectivity and a safe system.

The following non-limiting examples serve to illustrate the invention.

### EXAMPLES

The following examples illustrate this invention, but are not intended to limit the scope of the invention. Both batch and continuous examples are provided with the preferred method being continuous production. The batch examples given are intended to demonstrate some of the solvent systems used. All analysis reported here were run on a Hewlett-Packard Series II gas chromatograph, (GC), using a thermal conductivity detector. A Supelco 10 ft×⅛″ packed column using 5% Fluorcol on 60/80 Carbopack B support was used to effect product separation. The initial GC temperature of 30° C. was held for 7 minutes than-ramped at 50° C./min. to 200° C. and held for 25 minutes.

### Example 1

To a 400 cc Monel Parr autoclave equipped with agitator, thermocouple, dip tube, heating mantle, controller and cooling coils was charged: 400 g of C₆Cl₅F₉ isomer mixture solvent and 100 g of chlorotrifluoroethylene (CTFE). The system was heated to 70° C. An autogeneous pressure of 70 psig was recorded and a 15 psia pad of O₂ was maintained for a total system pressure of 85 psig. After 20 hours at 75° C. it was found that 92% of the CTFE had converted. The composition was determined by GC Analysis to be: 66.4% CClF₂COF, 21.8% CF₂O, 4.0% CClFO and 7.8% CTFE.

### Example 2

To a 650 cc Hastelloy, Parr autoclave equipped with agitator, thermocouple, dip tube, heating mantle, controller and cooling coils was charged: 600 g of C₆F₁₄ isomer mixture solvent and 93.6 g of chlorotrifluoroethylene (CTFE). The system was heated to 75° C. An autogeneous pressure of 80 psig was recorded and a 15 psia pad of O₂ was maintained for a total system pressure of 95 psig. After 22 hours at 75° C. it was found that 70% of the CTFE had converted. The composition was determined by GC Analysis to be: 66.5% CClF₂COF, 6.2% CF₂O, 3.9% CClFO and 29.4% CTFE.

### Example 3

To a 400 cc Monel Parr autoclave equipped with agitator, thermocouple, dip tube, heating mantle, controller and cooling coils was charged: 400 g of C₁₀ F₁₈ octadecafluorodecahydronaphthalene solvent and 100 g of CTFE. The system was heated to 80° C. An autogeneous pressure of 80 psig was recorded and a 15 psia pad of O₂ was maintained for a total system pressure of 95 psig. After 21 hours at 80° C. it was found that 94% of the CTFE had converted. The composition was determined by GC Analysis to be: 74.5% CClF₂COF, 14.3% CF₂O, 5.7% CClFO and 5.5% CTFE.

### Example 4

To a 400 cc Monel Parr autoclave equipped with agitator, thermocouple, dip tube, heating mantle, controller and cooling coils was charged: 400 g of C₆Cl₅F₉ isomer mixture solvent and 63.7 g of CTFE. The system was heated to 70° C. An autogeneous pressure of 66 psig was recorded and a 30 psia pad of O₂ was maintained for a total system pressure of 96 psig. After 8 hours at 70° C. it was found that 93% of the CTFE had converted. The composition was determined by GC Analysis to be: 76.1% CClF₂COF, 10.3% CF₂O, 6.5% CClFO and 7.1% CTFE.

### Example 5

To a 650 cc Hastelloy, Parr autoclave equipped with agitator, thermocouple, dip tube, heating mantle, controller and cooling coils was charged: 600 g of C₆Cl₅F₉ isomer mixture solvent and 54 g of (CTFE). The system was heated to 70° C. An autogeneous pressure of 38 psig was recorded and a 60 psia pad of O₂ was maintained for a total system pressure of 98 psig. After 4.5 hours at 70° C. it was found that all of the CTFE had converted. The composition was determined by GC Analysis to be: 80.9% CClF₂COF, 9.9% CF₂O, 9.2% CClFO and no CTFE.

### Example 6

To a 650 cc Hastelloy, Parr autoclave equipped with agitator, thermocouple, dip tube, heating mantle, controller and cooling coils was charged: 600 g of C₆Cl₅F₉ isomer mixture solvent and 55 g of (CTFE). The system was heated to 70° C. An autogeneous pressure of 40 psig was recorded and a 120 psia pad of O₂ was maintained for a total system pressure of 160 psig. After 2.5 hours at 75° C. it was found that all of the CTFE had converted. The composition was determined by GC Analysis to be: 85.3% CClF₂COF, 4.5% CF₂O, 8.6% CClFO and no CTFE.

### Example 7

To a 650 cc Hastelloy, Parr autoclave equipped with agitator, thermocouple, dip tube, heating mantle, controller and cooling coils was charged: 800 g of C₆Cl₅F₉ isomer mixture solvent and 71.3 g of (CTFE). The system was heated to 70° C. An autogeneous pressure of 30 psig was recorded and a 120 psia pad of O₂ was maintained for a total system pressure of 150 psig. After 3.0 hours at 70° C. it was found that all of the CTFE had converted. The composition was determined by GC Analysis to be: 83.2% CClF₂COF, 6.5% CF₂O, 10.3% CClFO and no CTFE.

### Examples 8-14

EXAMPLES No. 8 through 14 were run in the continuous mode using a continuously stirred tank reactor system. These examples were run using an 8% loading of CTFE in C₆Cl₅F₉ isomer mixture solvent. The above mixture is metered into a 650 cc Hastelloy, Parr autoclave equipped with agitator, thermocouple, dip tube, heating mantle, controller and cooling coils from a 1 liter stainless steel cylinder. Simultaneously, the same amount of material that is metered in is removed to a second 1 liter stainless steel receiver. The system is held at steady state temperature, pressure and feed rate until reaction equilibrium is reached.

Once equilibrium is achieved product conversion and distribution is determined. The effect of temperature and feed rate on reaction rate, conversion and selectivity was determined and data are given below.

| | Feed | | | | | | |
|---|---|---|---|---|---|---|---|
| | Rate | Reaction | O₂ | | | | |
| EXAMPLE | grams/ | Temp. | Pressure | Product Distribution: GC Area % | | | |
| Number | hour | ° C. | PSIG | CF₂O | CClFO | CClF₂COF | CTFE |
| 8 | 175 | 70 | 120 | 5.1% | 4.7% | 74.9% | 15.0% |
| 9 | 120 | 70 | 120 | 5.9% | 5.2% | 82.4% | 6.2% |
| 10 | 162 | 70 | 120 | 8.2% | 9.2% | 68.3% | 14.2% |
| 11 | 120 | 80 | 120 | 9.1% | 6.5% | 81.6% | — |
| 12 | 400 | 85 | 120 | 6.4% | 4.4% | 80.3% | 8.7% |
| 13 | 800 | 95 | 120 | 6.8% | 5.1% | 77.0% | 7.4% |
| 14 | 1490 | 105 | 120 | 5.3% | 7.2% | 62.2% | 20.1% |

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above and all equivalents thereto.

1. A process for the production of chlorodifluoroacetyl fluoride which comprises reacting a solvent solution of chlorotrifluoroethylene with oxygen in a reactor to form a product which comprises chlorodifluoroacetyl fluoride.

2. The process of claim 1 wherein the reacting is conducted in a continuous mode.

3. The process of claim 1 wherein the reacting is conducted in a batch mode.

4. The process of claim 1 further comprising the subsequent step of removing chlorodifluoroacetyl fluoride from the product.

5. The process of claim 1 further comprising the subsequent step of removing is residual solvent from the product, forming a mixture of the residual solvent with additional chlorotrifluoroethylene and recycling the mixture-to the reactor.

6. The process of claim 1 wherein the solvent is selected from the group consisting of halogenated butanes, halogenated hexanes, dimethyl cyclobutanes, octadecafluorodecahydronaphthalene, and combinations thereof.

7. The process of claim 1 wherein the solvent is selected from the group consisting of C₄FₓCl_{y} wherein x=1 to 10 and y=10-x; C₆FxCly wherein x=1 to 14 and y=14-x; C₆FₓCl_{y} wherein x=1 to 12 and y=12-x; and combinations thereof.

8. The process of claim 1 wherein the chlorotrifluoroethylene concentration in the solvent ranges from about 1% to about 30% by weight.

9. The process of claim 1 wherein the solvent solution is fed into the reactor at a rate which ranges from about 0.1 to about 3 times the reactor volume per hour.

10. The process of claim 1 wherein if the reactor has vapor space, the amount of chlorotrifluoroethylene in the vapor space is maintained at about less than 3 wt. %.

11. The process of claim 1 wherein the reaction is conducted at a temperature in the range of from about 20° C. to about 200° C.

12. The process of claim 1 wherein the oxygen partial pressure is maintained in the range of from about 10 psia about to 300 psia.

13. The process of claim 1 wherein the ratio of oxygen to chlorotrifluoroethylene ranges from about 0.01 to about 0.55 by weight.

14. A continuous process for the production of chlorodifluoroacetyl fluoride which comprises reacting a solvent solution of chlorotrifluoroethylene with gaseous oxygen with simultaneous agitation in a reactor to form a product which comprises chlorodifluoroacetyl fluoride.

15. The process of claim 14 wherein the reaction is conducted by continually feeding the solvent solution of chlorotrifluoroethylene into the reactor, wherein the reactor is pre-pressurized with oxygen.

16. The process of claim 14 further comprising the subsequent step of removing chlorodifluoroacetyl fluoride from the product.

17. The process of claim 14 further comprising the subsequent step of removing residual solvent from the product, forming a mixture of the residual solvent with additional chlorotrifluoroethylene and recycling the mixture to the reactor.

18. The process of claim 14 wherein the solvent is selected from the group consisting of halogenated butanes, halogenated hexanes, dimethyl cyclobutanes, octadecafluorodecahydronaphthalene, and combinations thereof.

19. The process of claim 14 wherein the solvent is selected from the group consisting of C₄FₓCl_{y} wherein x=1 to 10 and y=10-x; C₆FxCly wherein x=1 to 14 and y=14-x; C₆FₓCl_{y} wherein x=1 to 12 and y=12-x; and combinations thereof.

20. The process of claim 14 wherein the chlorotrifluoroethylene concentration in the solvent ranges from about 1% to about 30% by weight.

21. The process of claim 14 wherein the solvent solution is fed into the reactor at a rate which ranges from about 0.1 to about 3 times the reactor volume per hour.

22. The process of claim 14 wherein if the reactor has vapor space, the amount of chlorotrifluoroethylene in the vapor space is maintained at about less than 3 wt. %.

23. The process of claim 14 wherein the reaction is conducted at a temperature in the range of from about 20° C. to about 200° C.

24. The process of claim 14 wherein the oxygen partial pressure is maintained in the range of from about 10 psia about to 300 psia.

25. The process of claim 14 wherein the ratio of oxygen to chlorotrifluoroethylene ranges from about 0.01 to about 0.55 by weight.

26. A continuous process for the production of chlorodifluoroacetyl fluoride which comprises reacting a solvent solution of chlorotrifluoroethylene with gaseous oxygen with simultaneous agitation in a reactor to form a product which comprises chlorodifluoroacetyl fluoride; wherein the reaction is conducted by continually feeding the solvent solution of chlorotrifluoroethylene into the reactor, wherein the reactor is pre-pressurized with oxygen; and then subsequently removing chlorodifluoroacetyl fluoride from the product; wherein the solvent is selected from the group consisting of halogenated butanes, halogenated hexanes, dimethyl cyclobutanes, octadecafluorodecahydronaphthalene, and combinations thereof; wherein the chlorotrifluoroethylene concentration in the solvent ranges from about 1% to 30% by weight; wherein the solvent solution is fed into the reactor at a rate which ranges from about 0.1 to about 3 times the reactor volume per hour; wherein if the reactor has vapor space, the amount of chlorotrifluoroethylene in the vapor space is maintained at about less than 3 wt. %; wherein the reacting is conducted at a temperature in the range of from about 20° C. to about 200° C.; wherein the oxygen partial pressure is maintained in the range of from about 10 psia to about 300 psia; wherein the ratio of oxygen to chlorotrifluoroethylene ranges from about 0.01 to about 0.55 by weight.

27. The process of claim 26 further comprising the subsequent step of removing residual solvent from the product, forming a mixture of the residual solvent with additional chlorotrifluoroethylene and recycling the mixture to the reactor.

28. The process of claim 26 wherein the solvent is selected from the group consisting of C₄FₓCl_{y} wherein x=1 to 10 and y=10-x; C₆FxCly wherein x=1 to 14 and y=14-x; C₆FₓCl_{y} wherein x=1 to 12 and y=12-x; and combinations thereof.
